(19) United States
(12) Patent Application Publication
(10) Pub. No.: US 2008/0305157 A1
(43) Pub. Date: Dec. 11, 2008

(54) Encapsulation and separation of charged organic solutes inside catanionic vesicles

(75) Inventors: Douglas S. English, College Park, MD (US); Srinivasa Raghavan, College Park, MD (US); Philip R. DeShong, College Park, MD (US); Xiang Wang, College Park, MD (US); Emily J. Danoff, College Park, MD (US); Jaeho Lee, College Park, MD (US)

Correspondence Address: VENABLE LLP, P.O. BOX 34385, WASHINGTON, DC, 20043-9998, US

(73) Assignee: University of Maryland Office of Technology Commercialization, College Park, MD (US)

(21) Appl. No.: 12/155,758

(22) Filed: Jun. 9, 2008

Related U.S. Application Data
(60) Provisional application No. 60/942,728, filed on Jun. 8, 2007.

(51) Int. Cl. A61K 9/127; C11B 3/00; C12N 15/87; C12N 5/06

(52) U.S. Cl. 424/450; 554/124; 435/375; 435/458

(57) ABSTRACT

Catanionic vesicles including solute ion, methods for forming these, and methods of using these.

This application claims the benefit of U.S. Provisional Application No. 60/942,728, filed Jun. 8, 2007.

### BACKGROUND OF THE INVENTION

Embodiments of the invention relate to surfactant vesicles formed from mixtures of oppositely-charged single-tailed surfactants, commonly referred to as “catanionic” vesicles. For example, equilibrium vesicles that include a n anionic surface active agent and a cationic surface active agent are presented. Vesicles can be formed with a molar excess of one surfactant, so as to impart a net charge to the vesicle bilayer. The vesicles can sequester ionic molecules in aqueous solution, and separate charged molecules, such as organic molecules. For example, the vesicles can be used in neat form, e.g., composed of only two surfactants. Alternatively, an additional nonionic surfactant can be added for surface functionalization.

Good stability and long shelf life of vesicles bearing drugs or other molecules can be important. Conventional phospholipid vesicles formed by sonication or extrusion are essentially kinetically trapped nonequilibrium structures. Over time, these vesicles tend to fuse or rupture to form lamellar phases, and in the process, their contents are likely to be released.

### SUMMARY

Vesicles are single bilayer shells, which are composed of amphipathic molecules such as surfactants or detergents. As used in this disclosure, the term “vesicle” will be understood as referring to lamellar structures such as unilamellar or multilamellar tube, sphere, or onion-like structures, for example, “vesicle” can refer to unilamellar bilayer shells having a relatively small size, for example, having a diameter of about 50-1000 nanometers. The shells can form spontaneously in aqueous media from mixtures of surfactants. Most studies on solute encapsulation by vesicles have used vesicles made from two-tailed amphiphiles (lipids). The term “liposome” is distinguished for purposes of this disclosure as referring to vesicles formed from phospholipids.

A method for sequestering a solute ion within a catanionic vesicle according to the invention includes the following. The charge of the solute ion can be determined, and a catanionic vesicle having a net surface charge having sign opposite to the sign of the charge of the solute ion can be created. The catanionic vesicle can be combined with the solute ion, and the catanionic vesicle can be allowed to sequester the solute ion. The solute ion can be, for example, a biologically active compound, a human, animal, and/or plant pharmaceutical agent, a fluorescently active chemical, a cosmetic chemical, an agriculturally active chemical, a fertilizer, a nutrient, a pesticide, and/or an herbicide.

A method for sequestering a solute ion within a catanionic vesicle according to the invention includes the following. The charge of a solute ion in a solution can be determined. A cationic surfactant and an anionic surfactant can be added to the solution, with the concentrations of the cationic surfactant and the anionic surfactant in the solution selected to produce catanionic vesicles with a net surface charge having sign opposite of the sign of the charge of the solute ion. The catanionic vesicle can be allowed to sequester the solute ion.

A method for separating a solute ion from a bulk solution according to the invention includes the following. A catanionic surfactant vesicle can be administered to the bulk solution, so that an inner water pool and/or a bilayer of the catanionic surfactant vesicle sequesters the solute ion from the bulk solution. A selective mechanism capable of distinguishing the catanionic surfactant vesicles from the bulk solution can be used to separate from the bulk solution the catanionic surfactant vesicle that sequesters the solute ion in order to remove the solute ion from the bulk solution. The solute ion can have a charge. The sign of the charge of the solute ion can be identified. The catanionic surfactant vesicle can include the bilayer comprising a cationic surfactant and an anionic surfactant. The inner water pool can be separated from the bulk solution by the bilayer. The cationic surfactant and anionic surfactant including the bilayer can have a net surface charge. The net surface charge can have sign opposite to that of the solute ion; for example, the molar ratio of cationic surfactant to anionic surfactant can be selected, so that the net surface charge has sign opposite to that of the solute ion. The solute ion can be, for example, an atomic ion, a charged inorganic molecule, or a charged organic molecule. The selective mechanism can be, for example, size exclusion chromatography (SEC), affinity chromatography, and/or electrokinetic chromatography.

In an embodiment according to the present invention, an aqueous composition includes an aqueous environment and a catanionic surfactant vesicle. The catanionic surfactant vesicle can include a bilayer including a cationic surfactant and an anionic surfactant. The catanionic surfactant vesicle can include an inner pool separated from the aqueous environment by the bilayer. The solute ion can have a charge within the inner pool and/or the bilayer. The bilayer can have a net surface charge. The net surface charge can have sign opposite to that of the solute ion. The solute ion can be a metal, a dye, carboxyfluorescein, Lucifer yellow, Rhodamine 6G, Sulforhodamine 101, a drug, doxorubicin, a chemotherapeutic agent, a natural product, a peptide, an oligopeptide, a polypeptide, a nucleotide, an oligonucleotide, a polypeptide, DNA, RNA, derivatives of these, and combinations. The anionic surfactant can be alkyl sulfate, sodium octyl sulfate, sodium decyl sulfate, sodium dodecyl sulfate, sodium tetra-decyl sulfate, alkyl sulfonates, sodium octyl sulfonate, sodium decyl sulfonate, sodium dodecyl sulfonate, alkyl benzene sulfonates, sodium octyl benzene sulfonate, sodium decyl benzene sulfonate, sodium dodecyl benzene sulfonate, fatty acid salt, sodium octanoate, sodium decanoate, sodium dodecanoate, sodium salt of oleic acid, derivatives of these, and combinations. The cationic surfactant can be alkyl trimethylammonium halide, octyl trimethylammonium bromide, decyl trimethylammonium bromide, dodecyl trimethylammonium bromide, myristyl trimethylammonium bromide, cetyl trimethylammonium bromide, alkyl trimethylammonium tosylate, octyl trimethylammonium tosylate, decyl trimethylammonium tosylate, dodecyl trimethylammonium tosylate, myristyl trimethylammonium tosylate, cetyl tri-methylammonium tosylate, N-alkyl pyridinium halide, decyl pyridinium chloride, dodecyl pyridinium chloride, cetyl pyridinium chloride, derivatives of these and combinations. The cationic and/or the anionic surfactant can be SDS, DTAC, DTAB, DPC, DDAO, DDAB, SOS, AOT, derivatives of these, and combinations. The cationic surfactant can be a single alkyl chain surfactant and the anionic surfactant can be a single alkyl chain surfactant. The solute ion can be a cation having positive charge, and the cationic surfactant and anionic surfactant included in the bilayer can be in proportions to create a bilayer with a negative net surface charge. The solute ion can be an anion having negative charge, and the cationic surfactant and anionic surfactant included in the bilayer can be in proportions to create a bilayer with a positive net surface charge. The solute ion can be a deoxyribonucleic acid molecule, a ribonucleic acid molecule, a nucleotide, an oligonucleotide, a polynucleotide, a peptide, an oligopeptide, or a polypeptide. The aqueous composition can include catanionic surfactant vesicles at a high concentration (i.e., many vesicles per cubic centimeter volume), at an intermediate concentration, or at a low (i.e., dilute) concentration. The concentration of catanionic vesicles in the aqueous composition can be influenced by the application for which the aqueous composition is intended. For example, for a drug delivery application the concentration of catanionic vesicles in the aqueous composition can be influenced by the concentration of a therapeutic solute ion in the vesicles, the size of the vesicles (the selection of which can be influenced by, e.g., the need for the vesicles to permeate a biological membrane or barrier), and the appropriate overall concentration of therapeutic solute ion in the aqueous composition to be administered to a subject, e.g., a human, animal, or plant, in order to achieve a therapeutic effect.

In an aqueous composition according to the present invention, cationic surfactant vesicles can have a narrow distribution of diameters, a broad distribution of diameters, or a complex (e.g., multimodal) distribution of diameters. For example, a cationic surfactant vesicle can have a diameter in a range of from about 5, 10, 20, 30, 40, 50, 60, 70, 75, 80, 90, 100, 150, 200, 250, 500, 1000, 2000, and 5000 nanometers to about 5, 10, 20, 30, 40, 50, 60, 70, 75, 80, 90, 100, 150, 200, 250, 500, 1000, 2000, and 5000 nanometers.

The bilayer can include cationic surfactant and anionic surfactant in a molar ratio in a range of from about 9:1 to about 1:9, excluding a molar ratio of about 1:1. For example, the bilayer can include cationic surfactant and anionic surfactant in a molar ratio of from about 9:1, 8:2, 7:3, 6:4, 5.5:4.5, 5.1:4.9, 4.9:5.1, 4.5:5.5, 4:6, 3:7, 2:8, and 1:9 to about 9:1, 8:2, 7:3, 6:4, 5.5:4.5, 5.1:4.9, 4.9:5.1, 4.5:5.5, 4:6, 3:7, 2:8, and 1:9. For example, the bilayer can include cationic surfactant and anionic surfactant in a molar ratio in a range of from about 6:4 to about 8:2, in a range of from about 6:4 to about 7:3, of about 6:4, in a range of from about 2:8 to about 4:6, in a range of from about 3:7 to about 4:6, and of about 4:6. The cationic surfactant and the anionic surfactant can have a concentration in the external aqueous environment of less than about 5 wt %. For example, the cationic surfactant and the anionic surfactant can have a concentration in the external aqueous environment of from about 0.0001 wt % to about 3 wt %, for example, of from about 0.5 wt % to about 2 wt %, for example, of about 1 wt %. The solute ion can be present in the aqueous environment at an external concentration, the solute ion can be present in the vesicle at a sequestration concentration, and the ratio of the sequestration concentration to the external concentration can be greater than 1, for example, greater than or equal to 5. For example, from about 20% to about 75% of the solute ion present in the aqueous environment and in the catanionic surfactant vesicle can be sequestered in the catanionic surfactant vesicle. The encapsulation efficiency of the solute ion in the vesicle can be at least about 2%, for example, at least about 3%, greater than about 7%, or at least about 10%, 20%, 30%, 40%, 50%, 60%, 70%, 75%, 80%, 90%, or 95%. The percentage of solute adsorbed on the bilayer can be at least about 0.5%, for example, at least about 1%, 2%, 5%, or 16%. The ratio of the percentage of solute adsorbed on the bilayer to the encapsulation efficiency can be at least about 10%, for example, greater than 25%, at least about 50%, at least about 75%, at least about 90%, or at least about 95%.

The release of solute ion from a catanionic vesicle according to the present invention can occur over a range of time such that the half-life time of the release is from about 2, 5, 10, 20, 30, 40, 50, 60, 70, 80, 85, 90, 100, 120, 150, 200, and 500 days to about 2, 5, 10, 20, 30, 40, 50, 60, 70, 80, 85, 90, 100, 120, 150, 200, and 500 days.

A method of treating a living organism according to the invention can include administering catanionic surfactant vesicles including a therapeutically effective amount of a is solute ion to the organism. The catanionic surfactant vesicle can include a bilayer comprising a cationic surfactant and an anionic surfactant and an inner water pool separated from an aqueous environment by the bilayer. The inner water pool and/or the bilayer can include the solute ion. The solute ion can have a charge. The bilayer can have a net surface charge. The net surface charge can have sign opposite to that of the solute ion. The organism can be, for example a human, an animal, or a plant. The catanionic surfactant vesicles including the therapeutically effective amount of a solute ion can be administered orally or intravenously.

A method of introducing a nucleic acid into a cell according to the invention can include administering catanionic surfactant vesicles comprising a nucleic acid to the cell. The inner pool and/or the bilayer of the catanionic surfactant vesicle can include the nucleic acid. The bilayer can have a positive net surface charge. The nucleic acid can have a negative charge.

A kit according to the invention includes a premeasured amount of an anionic surfactant in a first labeled or unlabeled container and a premeasured amount of a cationic surfactant in a second labeled or unlabeled container. The premeasured amount of the anionic surfactant and the premeasured amount of the cationic surfactant can be selected, so that when the premeasured amounts are added to a predetermined amount of water containing a solute ion having a charge, catanionic surfactant vesicles are formed.

A kit according to the invention includes a mixture of an anionic surfactant and a cationic surfactant in a labeled or unlabeled container. The anionic surfactant and the cationic surfactant can be in a predetermined molar ratio in the mixture. The predetermined molar ratio can be selected, so that when the mixture is added to a predetermined amount of water containing a solute ion having a charge, catanionic surfactant vesicles are formed.

A kit according to the invention includes a mixture of an anionic surfactant, a cationic surfactant, and a solute ion having a charge in a labeled or unlabeled container. The anionic surfactant and the cationic surfactant can be in a predetermined molar ratio in the mixture. The predetermined molar ratio can be selected, so that when the mixture is added to a predetermined amount of water containing a solute ion having a charge, catanionic surfactant vesicles are formed.

### BRIEF DESCRIPTION OF THE DRAWINGS

FIG. 1 (left) presents a cartoon illustrating the favored formation of a double-tailed surfactant from oppositely charged monoalkyl surfactants. The cartoon at right represents the laminar structure formed by self-assembly of the double-tailed surfactant pairs and single-tailed unpaired surfactants.

FIG. 2 presents a ternary phase diagram showing the ideal conditions and solution dilutions (represented by blue lobes) for aqueous sodium dodecylbenzenesulfonate (SDBS) and cetyltrimethylammonium tosylate (CTAT) to form laminar vesicles.

FIG. 3 presents a cartoon illustrating a catanionic vesicle model showing the sequestration of solutes by electrostatic absorption and encapsulation both inside and outside the laminar structure.

FIG. 4 presents a graph representing the release of cargo solute from catanionic vesicles over a period of time.

FIG. 5 presents graphs and photographs illustrating the efficient separation of the two oppositely charged dyes—carboxyfluorescein (CF) and rhodamine G6 (RG6)—by size exclusion chromatography (SEC) using catanionic vesicles. Top panel (A) shows photographs depicting the efficient separation. The lower panels (B, C) show DLS and UV-vis absorbance measurements of the eluted fractions. The solid line in each is the DLS intensity, the one set of dotted lines represents the carboxyfluorescein (CF) absorbance and the other set of dotted lines represents the Rhodamine 6G (R6G) absorbance. In panel B, it can be seen that CF elutes with V+ vesicles and in panel C it is seen that R6G elutes with V− vesicles.

FIG. 6 presents the anionic dye carboxyfluorescein (CF) that is sequestered by catanionic vesicles. The upper panels show the efficient encapsulation of CF in positively charged vesicles (V+). The lower panels illustrate that far less dye is encapsulated in vesicles that are negatively charged.

FIG. 7 presents a graph representing cargo release as a function of time, R(t), from phospholipids (dotted line) and R(t) from catanionic vesicles (solid line).

FIG. 8 presents a graph of fluorescence intensity as a function of time for denaturation of catanionic vesicles and associated release of carboxyfluorescein.

FIG. 9 presents chemical structures of the solutes utilized in experiments to discussed.

FIG. 10 presents a graph representing the release of solute from cationic vesicles over time.

FIG. 11 presents graphs representing SANS data obtained for neat and for dye-containing V+ and V− catanionic vesicle samples.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without parting from the spirit and scope of the invention. All references cited herein are incorporated by reference as if each had been individually incorporated.

The invention contemplates methods for producing and using catanionic vesicles which are capable of selectively sequestering solute ions in an aqueous solution shared by the vesicles. The vesicles produced in accordance with this invention are comprised of a mixture of cationic and anionic surfactants. These surfactants are preferably single-tailed monoalkyl surfactants. As is known in the art, surfactants in general are a quite broad class of structurally diverse molecules. Surface active agents generally share common features; all surfactants are amphipathic molecules composed of one or more than one hydrophobic hydrocarbon region referred to as the “tail” region, and a hydrophilic, polar region referred to as the “head region” or “head group.” The amphipathic nature of these molecules is responsible for their behavior at and influence upon phase interfaces.

The term “solute ion” includes, for example, single atom ion species, multiple atom, low molecular weight ion species, and large molecules with net charge, such as polypeptide and polynucleotides, e.g., DNA and RNA, and generally excludes nonionic molecules, such as glucose. A solute ion can be sequestered in a given amount in the bilayer and in the inner water pool of a vesicle. Sequestration concentrations according to the invention are high.

Vesicles have a number of important utilities, including chemical and biochemical applications. For example, vesicles are useful in performing biochemical assays which involve the storage or sequestration of biological materials such as enzymes or their substrates, by allowing for controlled protection and release of a sequestered substance. It is also possible to incorporate a reaction substrate into a vesicle membrane bilayer for presentation on the surface of the vesicle. Both vesicles and liposomes are of considerable interest in the controlled release and targeted delivery of pharmaceutically active agents in humans, animals, and plants, for example, in the fields of drug delivery, agrochemicals, and cosmetics. For example, vesicles can be useful for the targeted delivery of pesticides, fertilizers, and nutrients in agriculture. For example, loading a medication into vesicles or liposomes can serve to protect the medication from degradation or dilution in the blood. Vesicles are also useful in preparing models for the study of photosynthesis and membrane phenomena, by incorporating the appropriate molecules into the vesicle membrane in order to induce electron transfers and/or establish proton gradients.

Approaches to improving vesicle stability and encapsulation properties, for example, of conventional phospholipid vesicles, include changing bilayer compositions or by using micron-sized vesicles.

A simple attractive alternative to phospholipid vesicles in some applications may be offered by surfactant vesicles, formed by mixing single-tailed cationic and anionic surfactants to form “catanionic” vesicles. Catanionic surfactant vesicles have several advantages over conventional phospholipid vesicles. For example, they form spontaneously without the need for additional sonication or extrusion, they have an extremely long shelf life, and the raw materials are inexpensive compared to synthetic or purified phospholipids. Catanionic vesicles can be spontaneously generated when the individual surfactants are mixed with water in the right proportion. Vesicle formation is thus quicker and easier compared to phospholipid vesicles, since extrusion or sonication steps are not required. Furthermore, the required materials are common surfactants that are cheaper than purified or synthetic phospholipids. Catanionic vesicles tend to be stable for very long periods of time, although it is not clear whether catanionic vesicles are truly equilibrium structures.

Single-tailed amphiphiles can form vesicles. Simple mixtures of cationic and anionic surfactants, which can be referred to as “catanionic” systems, can spontaneously give rise to unilamellar vesicles in water. Several catanionic vesicle-forming systems have been studied with respect to their phase behavior. However, little is known about the ability of catanionic vesicles to encapsulate and retain organic molecules.

For application of catanionic vesicles as storage and delivery agents of small molecules, the critical issue is their ability to encapsulate molecules. In particular, a key unanswered question is: how do catanionic surfactant vesicles compare to conventional phospholipid vesicles in regards to encapsulation efficiency and membrane permeability? Despite the extensive literature on catanionic vesicles, there is surprisingly little information on their encapsulating abilities or the permeability of their bilayers. The few studies that have explored encapsulation with well-characterized catanionic vesicles focused principally on the entrapment of glucose. The ability of catanionic vesicles to entrap and encapsulate solutes, especially ionic molecules, remains by and large untested.

An example of a catanionic vesicle-forming system is the cetyltrimethylammonium tosylate—sodium dodecylbenzenesulfonate (CTAT/SDBS) system. CTAT/SDBS vesicles can be unilamellar and fairly monodisperse, with radii of 60-80 nm.

Kaler reported that catanionic vesicles formed from CTAT and SDBS were able to encapsulate glucose but did not provide any quantitative data on the subject. See, C. Tondre and C. Caillet, Adv. Coll. Inter. Sci, 2001, 93, 115-134; E. W. Kaler, A. K. Murthy, B. E. Rodriguez and J. A. N. Zasadzinski, Science, 1989, 245, 1371-1374. Later, Kondo et al. studied glucose entrapment in vesicles formed from the surfactants didodecyldimethylammonium bromide (DDAB) and sodium dodecyl sulfate (SDS) catanionic vesicle system. They found maximum encapsulation of ˜7.9% of the initial glucose solution. See, Y. Kondo, H. Uchiyama, N. Yoshino, K. Nishiyama and M. Abe, Langmuir, 1995, 11, 2380-2384. Bhattacharya studied vesicles formed from hybrid (bolaphile/amphiphile) ion-pairs and found that they were able to entrap riboflavin, but only with 1-2% encapsulation values. See, S. Bhattacharya, S. M. De and M. Subramanian, J. Org. Chem., 1998, 63, 7640-7651.

Several new uses for spontaneously generated catanionic vesicles are presented as the embodiments of the instant invention. For example, catanioic vesicles can be used to sequester and separate charged solutes from solution and to sequester such solutes for long periods of time. For example, catanionic vesicles with a net positive charge can be used to sequester negatively charged (anionic) solute molecules for extended periods, and catanionic vesicles created with a net negative charge can be used to sequester positively charged (cationic) solutes. Catanionic vesicles can be used in a process for separating oppositely charged molecules in aqueous solution. Separation can be quick and efficient and can be effective for molecules of differing or similar mass provided they have opposite net charges. Because the vesicles can be stable over long periods and can store sequestered molecules for long periods, such catanionic vesicles can be useful as storage and delivery mechanism, for example, in applications such as drug delivery or delivery of reagents in diagnostic applications in vivo or in vitro.

In an embodiment according to the present invention, vesicles are prepared in aqueous solution from simple, single-chain surfactants. The vesicles contain at least one anionic surfactant and at least one cationic surfactant, and are formed spontaneously in solution by combining aqueous solutions of the anionic and cationic surfactants. The resulting vesicles are equilibrium vesicles, i.e., they are highly stable over time. Alternatively, the dry cationic and anionic surfactants can be dissolved together. Regardless of the preparation technique, unilamellar vesicles spontaneously form. The single-tailed, anionic surfactant preferably comprises an amphipathic molecule having a C₆ to C₂₀ hydrocarbon tail region and a hydrophilic, polar head group. The headgroup on the anionic surfactant is preferably selected from sulfonate, sulfate, carboxylate, benzene sulfonate and phosphate. The single-tailed, cationic surfactant preferably comprises an amphipathic molecule having a C₆ to C₂₀ hydrocarbon tail region and a hydrophilic polar head group. The head group on the cationic surfactant can include, for example, a quaternary ammonium group, a sulfonium group, or a phosphonium group.

The single-tailed surface active agents useful in the practice of embodiments of the invention are relatively simple molecules. Such single-chain surfactants are inexpensive and are readily available in bulk. The use of simple, readily available surfactants lends an economic attractiveness to the practice of the present invention.

In accordance with various embodiments of the invention, methods for creating and using vesicles which specifically encapsulate and sequester either cationic or anionic solutes are discussed. Methods for using vesicles so created as a means for separating out charged solutes from a mixed solution, and of separating similarly sized but oppositely charged solutes are provided.

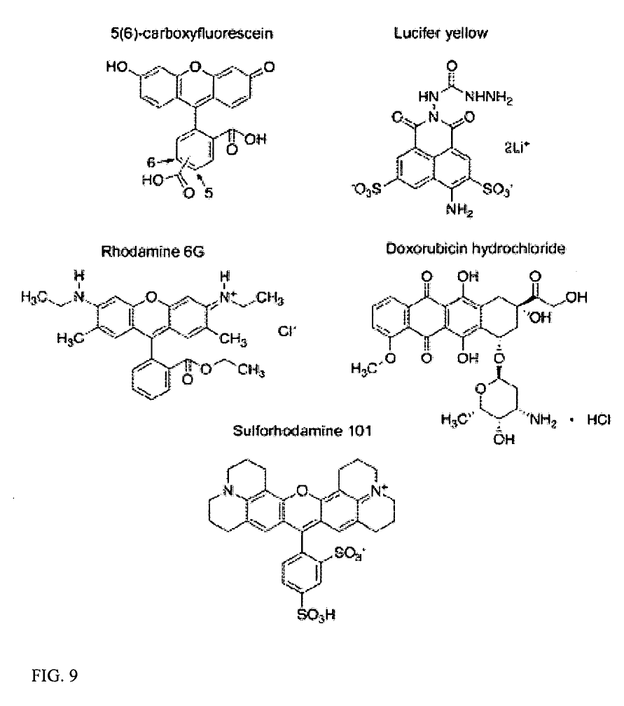

Catanionic vesicles can efficiently encapsulate solutes that are of the opposite charge from the vesicles, and can retain these molecules for long periods of time. For example, catanionic vesicles with a molar excess of the cationic surfactant cetyltrimethylammonium tosylate (CTAT) efficiently capture the anionic dye 5(6)-carboxyfluorescein (CF), and retain it for very long periods of time (half life t_{1/2} of 84 days). For example, anionic and cationic organic molecules such as those shown in FIG. 9 can all be sequestered in catanionic vesicles as indicated, for example, in Table 1. Table 1 presents apparent encapsulation efficiency (ε) values and vesicle radius for four dyes and the drug doxorubicin in the V⁺ and the V⁻ CTAT/SDBS catanionic vesicle systems. Catanionic surfactant vesicles can be highly efficient for the capture and long-term storage of organic solutes that have a charge opposite to that of the vesicles. For example, negatively charged catanionic vesicles can encapsulate the cationic anti-cancer drug, doxorubicin. Strong, specific, charge-mediated interactions can occur between catanionic vesicles and solutes. These interactions can be harnessed for the efficient separation of oppositely-charged solutes from a solute mixture using only separation techniques, such as conventional, gravity-driven, size exclusion chromatography (SEC).

TABLE 1

| | ε (Apparent Encapsulation | | Vesicle Radius after | |
|---|---|---|---|---|
| | Efficiency) | | SEC by DLS(nm) | |
| Probe | CTAT-Rich | SDBS-Rich | CTAT-Rich | SDBS-Rich |
| Molecule | V⁺ | V⁻ | V⁺ (81 ± 13) | V⁻ (98 ± 6) |
| CF | 24 ± 4% | 1.0 ± 0.4% | 87 ± 5 | 91 ± 8 |
| LY | 40 ± 20% | 4% | 208 ± 18 | 96 ± 3 |
| SR 101 | 32.8% | 8.2% | 122 ± 38 | 84 ± 7 |
| R6G | 0.07 ± 0.1% | 72 ± 3% | 156 ± 24 | 109 ± 16 |
| Dox | 0% | 55% | 143 ± 32 | 93 ± 4 |

Single-chain surfactants useful for embodiments of the invention are amphipathic molecules having a single, hydrophobic tail region, and a single, polar head region. The hydrocarbon tail region of the surfactant molecule can be aliphatic. For example, the tail region of the surfactant molecule can include a hydrocarbon chain having between 6 and 20 carbon atoms, which can be saturated, unsaturated, or substituted, provided that the essentially hydrophobic character of the tail region is preserved. When the length of the tail region exceeds about 18 carbon atoms, the temperature of the aqueous surfactant solution can be increased to maintain solubility.

The charge of the polar head group determines the charge of the surface active agent. In an embodiment according to the invention, vesicles are composed of at least one anionic surfactant and at least one cationic surfactant. Moieties comprising the polar head group in the anionic surfactant can include sulfonate, sulfate, carboxylate, benzene sulfonate, and/or phosphate groups. Exemplary anionic, single-chain surface active agents can include, for example, alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents can include, for example, alkyl trimethylammonium halides, alkyl trimethylammonium tosylates, and N-alkyl pyridinium halides.

Alkyl sulfates can include sodium octyl sulfate, sodium decyl sulfate, sodium dodecyl sulfate, and sodium tetra-decyl sulfate. Alkyl sulfonates can include sodium octyl sulfonate, sodium decyl sulfonate, and sodium dodecyl sulfonate. Alkyl benzene sulfonates can include sodium octyl benzene sulfonate, sodium decyl benzene sulfonate, and sodium dodecyl benzene sulfonate. Fatty acid salts can include sodium octanoate, sodium decanoate, sodium dodecanoate, and the sodium salt of oleic acid.

Alkyl trimethylammonium halides can include octyl trimethylammonium bromide, decyl trimethylammonium bromide, dodecyl trimethylammonium bromide, myristyl trimethylammonium bromide, and cetyl trimethylammonium bromide. Alkyl trimethylammonium tosylates can include octyl trimethylammonium tosylate, decyl trimethylammonium tosylate, dodecyl trimethylammonium tosylate, myristyl trimethylammonium tosylate, and cetyl trimethylammonium tosylate. N-alkyl pyridinium halides can include decyl pyridinium chloride, dodecyl pyridinium chloride, and cetyl pyridinium chloride.

Surfactants that can be used to form catanionic vesicles according to the present invention include, for example, SDS, DTAC, DTAB, DPC, DDAO, DDAB, SOS, and AOT.

It will be understood that the above listings are representative rather than exhaustive. It will also be appreciated that many surfactants are available as polydisperse mixtures rather than as homogeneous preparations of a single surfactant species, and such mixtures are also contemplated by this invention.

In a method according to the invention, catanionic vesicles are spontaneously formed in aqueous solution without the need for mechanical or chemical treatments beyond mild stirring to aid in mixing and dissolving the two surfactants. Unilamellar vesicles can be formed spontaneously upon combining an aqueous solution of a single-tailed, anionic surfactant with an aqueous solution of a single-tailed, cationic surfactant. The resulting catanionic vesicles can be equilibrium vesicles, i.e., they can be thermodynamically stable over extended time periods, such as up to one year. Catanionic vesicles so prepared can be capable of withstanding freeze-thaw cycles without disruption or release of their contents.

In a method according to the invention, aqueous solutions of the cationic and anionic surfactants are prepared from surfactant salts or by diluting concentrated surfactant solutions to the desired stock concentrations. A stock solution of the anionic surfactant can be combined with a stock solution of the cationic surfactant, and the resulting reaction solution can be gently mixed. Catanionic vesicles can form immediately and spontaneously in the reaction solution upon combination of the stock solutions. The presence of vesicles in the resulting reaction solutions can be confirmed by techniques such as, for example, quasi-elastic light scattering, freeze-fracture or cryogenic transmission electron microscopy, small angle neutron scattering (SANS), and/or glucose entrapment experiments. Catanionic vesicles can also be formed by mixing cationic and anionic surfactant salts prior to the addition of and mixing with an aqueous solutions. For example, catanionic vesicles can be formed directly by adding water to solubilize the dry catanionic and anionic surfactants prepared at the appropriate ratios. Vesicles in aqueous solution can be recovered or concentrated using techniques such as filtration and/or centrifugation.

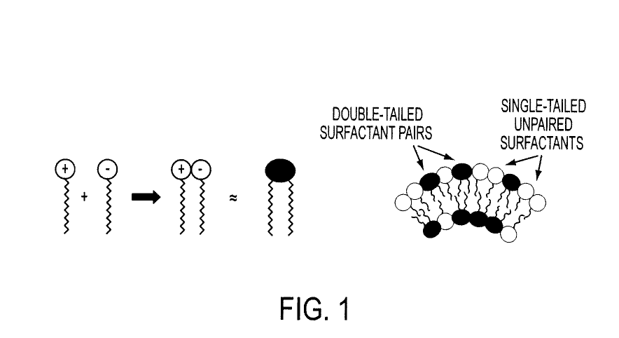

Vesicle formation can be facilitated by the formation of ion-pair amphiphiles as shown in FIG. 1. These ion-pairs have geometries which favor formation of a lamellar or bilayer structure. In the presence of an excess of one surfactant, the unpaired surfactant can promote vesicle formation by inducing a spontaneous curvature through non-ideal mixing in which the excess unpaired surfactants segregate to the outer leaflet of the vesicle bilayer, see FIG. 1. The positioning of the majority of unpaired surfactant to the bilayer outer leaflet results in a high surface charge on the vesicle exterior. The exterior charges provide binding sites for oppositely charged solute molecules in solution and can promote high loading capacity of catanionic surfactant vesicles.

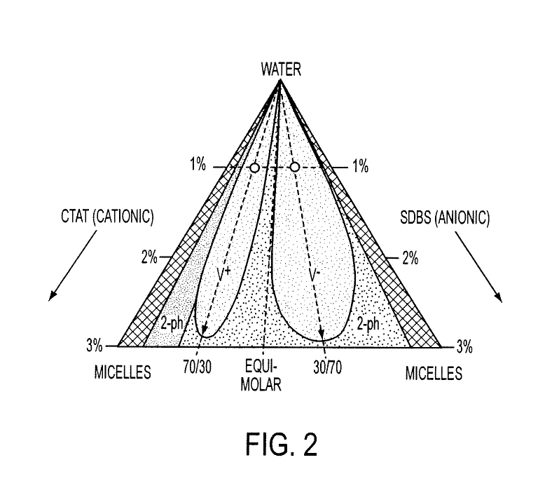

Catanionic vesicles are presently understood to spontaneously form in mixtures having an excess of one surfactant. FIG. 2 shows a ternary phase diagram for water, cetyltrimethylammonium tosylate (CTAT) and sodium dodecylbenzenesulfonate (SDBS). Two lobes exist in the water-rich corner of the phase diagram where stable vesicles are known to spontaneously form.

The term “sequestration” is used to refer to the incorporation of material from the aqueous phase of the general mixture into the aqueous solution within the interior of the vesicle space and/or into the internal and/or external bilayer leaflets of the shell of the vesicle. Sequestration is understood to be chiefly governed by electrostatic interactions between the excess surfactant, i.e., the surfactant present in greater concentration in the vesicle, and an is oppositely charged ionic solute (cargo) molecule. The ability of a vesicle to sequester ionic solutes depends upon the net charge of the vesicle. Net charge is a function of the molar ratio of the two surfactants that form the catanionic vesicle. For instance, if the anionic surfactant is in two-fold molar excess, then roughly half of the anions will be paired with a cationic surfactant molecule to form an ion-pair amphiphile. The remaining anions, roughly half of the original number, will be unpaired and present primarily on the external leaflet of the bilayer of the vesicle. Hence, the spontaneously formed vesicles will have a net external charge that is proportional to the concentration of the anionic surfactant. The unpaired surfactants form electrostatic binding sites for ionic solutes. The ratio of cationic to anionic molecules in the vesicles of the invention may vary within broad limits while maintaining the features of stability and spontaneous formation. Vesicles have been characterized which have an anionic surfactant to cationic surfactant ratio ranging between 1:9 and 9:1. The vesicle charge and size, and other vesicle characteristics, vary considerably over this range. However, combining equimolar amounts of anionic surfactant and cationic surfactant as stock solutions can result in the formation of insoluble precipitates or undesirable lamellar structures.

In an embodiment according to the invention, the sequestration of anionic solutes is accomplished by creating vesicles with a net surface positive charge. In an embodiment according to the invention, the sequestration of cationic solutes is accomplished by creating vesicles with a net surface negative charge. Imparting a surface charge on the vesicles can be accomplished by varying the proportion of cationic to anionic surfactants used in creating the vesicles. For example, if a larger proportion of the total surfactant in the final vesicles is of the anionic surfactant species, the vesicle surface can have a net negative charge, the magnitude of which may be precisely controlled. Negatively-charged vesicles can be prepared by increasing the amount of anionic surfactant relative to the amount of cationic surfactant in the reaction solution, either by adding a greater volume of the anionic solution or by adding a more concentrated anionic solution. The magnitude of the negative surface charge on the final vesicles can depend upon the amount by which the anionic surfactant species is greater in abundance than the cationic surfactant species in the reaction solution. Conversely, a net positive charge can be imparted to the surfaces of the vesicles by adding the cationic species in excess of the anionic species. Obtaining a desired surface charge on the membrane of the vesicles can thus be achieved by controlling the relative proportions of anionic and cationic surfactant.

Examples of materials which can be sequestered by vesicles include dyes, pharmaceuticals and components of cosmetics and detergents. The practitioner will realize that this list is nowhere near exhaustive of the sorts of material which may be sequestered by the vesicles according to the current invention.

In an embodiment according to the invention, sequestration of material in vesicles is achieved as follows. The water soluble ion to be sequestered can be in the solution prior to the creation of the vesicles, so that when the surfactants are mixed together with the ionic solute, the vesicles spontaneously form and the ionic solute is incorporated into and onto the vesicles.

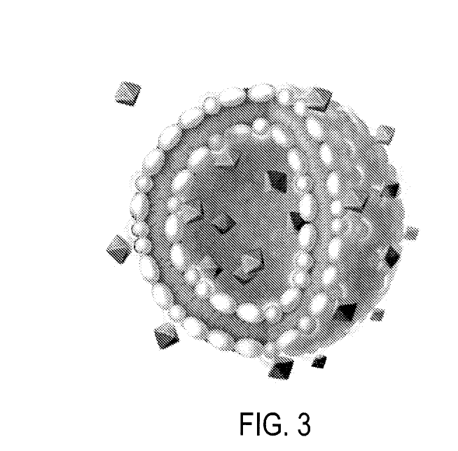

In an alternative embodiment according to the invention, sequestration of material in vesicles is achieved as follows. The vesicles can be pre-formed, and the ionic substance to be sequestered can then be added to the solution in which the vesicles are suspended. The ionic substance will then be incorporated through electrostatic interactions with the bilayer exterior and will be present primarily on the vesicle exterior as illustrated in FIG. 3 (where molecules of the ionic substance are illustrated as octahedra). This method of vesicle preparation and sequestration can allow for gentle yet efficient sequestration of an aqueous phase to take place, as the surfactant stock solutions are combined without mechanical or chemical perturbations from the final vesicle composition or structure.

The size and curvature properties (shape) of catanionic vesicles formed according to embodiments of the invention can vary depending upon factors such as the length of the hydrocarbon tail regions of the constituent surfactants and the nature of the polar head groups. For example, the diameter size of vesicles according to the invention can be between 10 and 250 nanometers, for example, between 30 and 150 nm. The vesicle size can be influenced by selecting the relative lengths of the hydrocarbon tail regions of the anionic and cationic surfactants. For example, large vesicles, e.g., vesicles of 150 to 200 nanometers diameter, can be formed when there is disparity between the length of the hydrocarbon tail on the anionic surfactant and the hydrocarbon tail on the cationic surfactant. For example, large vesicles can be formed when a C₁₆ cationic surfactant solution is combined with a C₈ anionic surfactant solution. Smaller vesicles can be produced by using anionic and cationic surfactant species of which the lengths of the hydrocarbon tails are more closely matched. The permeability characteristics of vesicles according to the present invention can be influenced by the nature of the constituent surfactants, for example, the chain length of the hydrocarbon tail regions of the surfactants. Longer tail lengths on the is surfactant molecules can decrease the permeability of the vesicles by increasing the thickness and hydrophobicity of the vesicle membrane (bilayer). For example, the control of reagent and substrate permeation across vesicle membranes can be an important parameter when using the vesicles as microreactors.

In an embodiment, catanionic vesicles according to the invention can be used for gently separating charged species from a solution. For example, charged species can be removed from solution by introducing a vesicle of opposite charge into the solution containing the target ion(s) to be removed, or by causing the spontaneous formation of vesicles in the solution of the target ion(s) by adding properly proportioned anionic and cationic surfactants to induce the spontaneous formation of vesicles with charge of sign opposite to that of the target ion(s), so that the target ion(s) are spontaneously sequestered in the vesicles. Once the vesicles have sequestered the target ion(s), the vesicles and ion(s) can be removed from the solution by separation techniques such as size exclusion chromatography (SEC), affinity chromatography, electrokinetic chromatography, and/or another separation technique.

For example, in a method according to the present invention catanionic vesicles are used to separate ions having charge of opposite signs from each other in solution. Separation can be achieved by mixing vesicles with a solution containing a number of different charged species. Vesicles having a charge of sign opposite to the target ion(s) can be formed as described herein in the solution with the target ions or the vesicles can be created separately and introduced to the solution after the vesicles have formed. The vesicles can then spontaneously sequester the target ion(s) having a charge of sign opposite to the net surface charge of the vesicles. After the vesicles have sequestered the ions, they can be separated from the solution by a mechanism capable of selectively separating vesicles. For example, separation techniques such as size exclusion chromatography (SEC), affinity chromatography, electrokinetic chromatography, and/or another separation technique can be used to separating out the target-ion-laden vesicles from the rest of the solution.

In an embodiment according to the invention, catanionic vesicles are used to store ionic solutes for extended period of time. The catanionic vesicles can be stable for long periods, so that material sequestered by the vesicles remains sequestered for extended periods of time. Therefore, catanionic vesicles according to the invention can be used to enhance the shelf life of sequestered chemicals by shielding them from the outside environment.

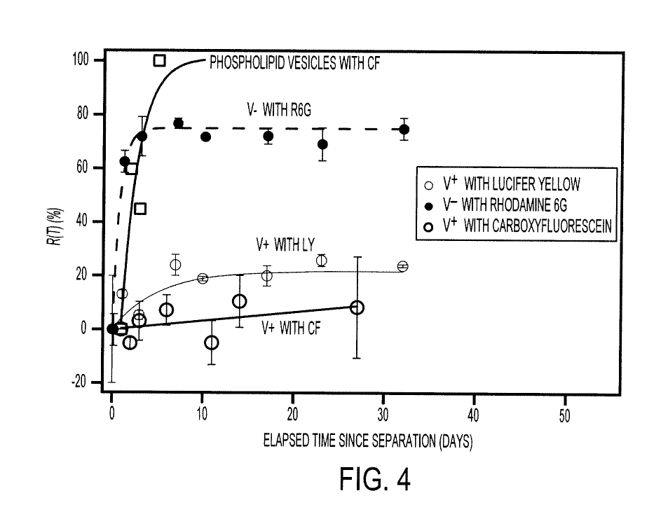

In an embodiment according to the invention, catanionic vesicles are used as slow release delivery mechanisms for substances such as drugs, agricultural chemicals, dyes, proteins, DNA vectors, plasmids, and organic molecules, e.g., bivalent organic biomolecules. Release times of the sequestered substance, for example, organic molecules, can be long, for example, on the order of weeks or months, as illustrated by FIG. 4.

### EXAMPLES

### Example 1

Catanionic Vesicles Formed from Cetyltrimethylammonium Tosylate (CTAT) and Sodium Dodecylbenzenesulfonate (SDBS)

The surfactants CTAT, SDBS, and Triton X-100 were purchased from Aldrich Chemicals. The fluorescent dyes CF, sulforhodamine 101 (SR 101), and Lucifer yellow (LY) were purchased from Molecular Probes, while the dye rhodamine 6G (R6G) and the chemotherapeutic drug, doxorubicin hydrochloride (Dox) were purchased from Fluka. All materials were used without further purification. The dry surfactants, CTAT and SDBS, were stored in a desiccator to prevent water absorption.

Vesicle samples were prepared at two different surfactant compositions, 7:3 and 3:7 w/w CTAT to SDBS, which are denoted as V⁺ and V⁻, respectively. V⁺ refers to the excess positive charge on the vesicle bilayers when there is an excess of CTAT, and likewise, V⁻ refers to vesicles with a net negative charge due to an excess of SDBS. All samples were prepared at a total surfactant concentration of 1 wt. %. The surfactants were weighed and mixed with deionized water by gentle stirring, and then allowed to equilibrate at room temperature for at least 48 h.

FIG. 2 shows the water-rich corner of the ternary phase diagram for mixtures of CTAT, SDBS, and water. Catanionic vesicles are present at compositions within the two lobes on either side of the equimolar line. Vesicle samples were prepared by weighing and mixing the two surfactants in water, followed by gentle stirring. For large unilamellar vesicles (LUV) of EYPC an extrusion method was used.

Vesicle sizes in solution were monitored using dynamic light scattering (DLS) on a Photocor-FC instrument. The light source was a 5 mW laser at 633 nm and the scattering angle was 90°. A logarithmic correlator was used to obtain the autocorrelation function, which was analyzed by the method of cumulants to yield a diffusion coefficient. The apparent hydrodynamic size of the vesicles was obtained from the diffusion coefficient through the Stokes-Einstein relationship. The intensity (total counts) of the signal was also recorded for each sample.

Small angle neutron scattering (SANS) experiments were conducted on the neat vesicles as well as vesicle-solute mixtures to probe whether there were any changes in vesicle size or bilayer integrity caused by the solutes. All samples for SANS experiments were prepared using deuterium oxide (99% D, from Cambridge Isotopes) in place of water. The measurements were made on the NG-7 (30 m) beamline at NIST in Gaithersburg, Md. Neutrons with a wavelength of 6 Å were selected. Two sample-detector distances of 1.33 m and 13.2 m were used to probe a wide range of wave vectors from 0.004-0.4 Å⁻¹. Samples were studied in 2 mm quartz cells at 25° C. The scattering spectra were corrected and placed on an absolute scale using calibration standards provided by NIST. The data are shown as the radially averaged intensity I (minus the background) versus the wave vector q=(4π/λ) sin(θ/2), where λ is the wavelength of incident neutrons and θ is the scattering angle.

### Example 2

Efficiency of Encapsulation of Solute Ions in Catanionic Vesicles Formed from Cetyltrimethylammonium Tosylate (CTAT) and Sodium Dodecylbenzenesulfonate (SDBS)

We studied the apparent encapsulation efficiency of CF in catanionic vesicles at two different CTAT/SDBS compositions, which are pinpointed in the phase diagram (FIG. 2). The first sample falls in the CTAT-rich vesicle lobe and consists of 1 wt % total surfactant with a 7:3 w/w of CTAT to SDBS. The vesicles in this case are denoted by V⁺ since they have a molar excess of the cationic surfactant. The second sample falls in the SDBS-rich vesicle lobe and it is a 3:7 w/w mixture of CTAT to SDBS at 1 wt % total surfactant. These vesicles are denoted by V⁻. The apparent encapsulation efficiency reflects contributions from dyes that are adsorbed to the bilayer or captured in the inner water pool. Therefore the actual encapsulation efficiency for dye in the V⁺ water pool is ca. 5%.

For the determination of encapsulation efficiencies, vesicles were prepared using aqueous solutions containing 1.0 mM carboxyfluorescein (CF) dye. The formation of CTAT-rich vesicles (7:3 CTAT to SDBS w/w) appeared to be inhibited at CF concentrations of 5 mM or greater. High concentrations of CF (and similarly, other solutes) tended to disrupt the vesicles and lead to precipitation over time. Vesicle stability appeared to be unaffected when the solute concentration is kept below 5 mM, and at these concentrations solute encapsulation does occur. CF is a trianionic fluorescent dye at a pH above 6.9; its structure is shown in FIG. 9. For CF, a pH ˜9 was required in order to fully dissolve the dye, and the stock solutions were adjusted accordingly. The CF/CTAT/SDBS solutions were stirred for 15-30 min and the resulting vesicle solutions were allowed to equilibrate in the dark at room temperature for at least 48 h. Dynamic light scattering (DLS) was then used to confirm vesicle formation. SDBS-rich vesicles (e.g., 3:7 CTAT to SDBS w/w) could be prepared with higher CF concentrations (up to ca. 100 mM); however CF encapsulation in these vesicles was reduced relative to that of CTAT-rich vesicles and in many cases CF encapsulation by SDBS-rich vesicles (i.e., the formation of distinct bands in the chromatography column, see below) was not achieved.

The apparent encapsulation efficiencies of the two vesicle preparations, V⁺ and V⁻, were also evaluated for the other solute molecules shown in FIG. 9. A solute concentration of 1 mM was used in all cases. Lucifer yellow (LY) is dianionic in water and Sulforhodamine 101 (SR101) is monanionic. Rhodamine 6G (R6G) possesses a quaternary amine, is cationic at all pH, and was chosen for its structural similarities with CF. Doxorubicin hydrochloride (Dox) is a cationic drug with a pKa of ˜7.619 that has been used to treat a variety of cancers. The toxic side effects of Dox have been shown to be reduced if it is delivered using liposomes. In each case, vesicles were prepared using aqueous solutions of the solute at a concentration of 1 mM. The solute/CTAT/SDBS mixtures were stirred for 30-60 min, or overnight, and the resulting vesicle solutions were allowed to equilibrate in the dark at room temperature for at least 48 h. Thereafter, the samples were passed through a 25 mm syringe filter (0.45 μm mesh) to remove any impurities. Dynamic light scattering was conducted to confirm vesicle formation and to measure the average vesicle size.

To measure the apparent encapsulation efficiency ε, size exclusion chromatography (SEC) was used to separate the free solute from that encapsulated by the vesicles. A 2×25 cm column packed with Sephadex G50 resin (medium mesh, Amersham Biosciences) or a 1.3 cm×21 cm SEC column packed with Sephadex G50 resin (medium mesh, from Amersham Biosciences) was used. An aliquot of the vesicle-solute sample was run through the SEC column.

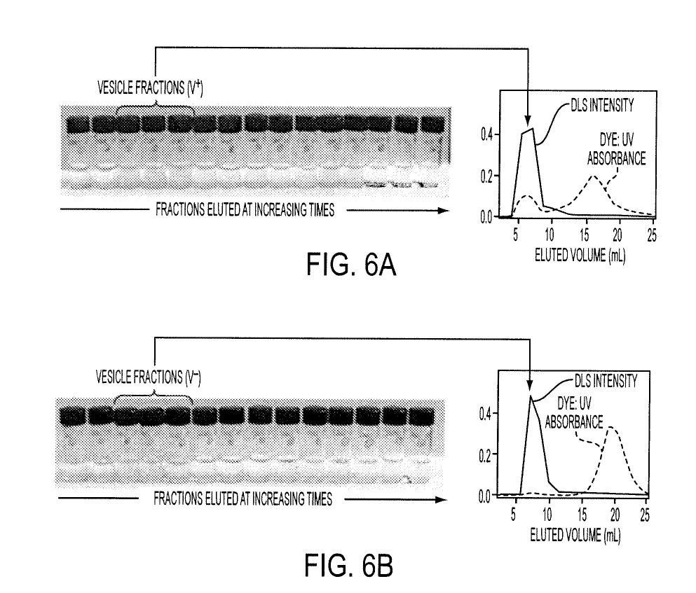

During elution, vesicle solutions divided into bands that were visible with the naked eye or by UV lamp, e.g., in the case of CF dye containing vesicles, two clear bands, the leading band containing the dye-bearing vesicles and the second band containing the free dye. During elution, fractions were collected and analyzed, and a series of such fractions for a typical experiment is shown in FIG. 6 (the solute in FIG. 6 is CF). The band containing the vesicles was collected for further studies of vesicle leakage, as described below. Dynamic light scattering was used to determine which of the eluted fractions contained vesicles, and the vesicles were consistently found to elute at 5.5 ml, total elution volume.

In the case of the CF dye containing vesicles, the DLS results from the leading band in the SEC column always gave values for hydrodynamic radius and total scattering intensity that were consistent with the presence of vesicles. Initial V⁻ samples, prior to SEC, were found to have an average radius of 76±5 nm, which was constant throughout the dilute surfactant range of 1.0% to 0.004% total surfactant concentration. This is consistent with the phase diagram in FIG. 2. V⁺ samples were also studied after elution from the SEC column and the measured average radius was 90±5 nm.

The amount of solute in each fraction was determined using UV-vis spectroscopy (Hitachi U-3010 Spectrometer). The encapsulation efficiency (ε) value is defined as the amount of encapsulated solute relative to the total initial amount of solute:

$$ɛ = \frac{{V}_{f} ( {A}_{f 1} + {A}_{f 2} + … )}{{V}_{i} {A}_{i}} . \qquad ( 1 )$$

V designates volume, A designates absorbance, i denotes initial values taken from the original preparation, and f denotes values taken from the fractions eluted from the SEC column shown by dynamic light scattering to contain vesicles. Thus, the value of ε gives percentage of dye that is captured by the vesicles during their preparation. To avoid artifacts in UV-Vis spectroscopy from light scattering or from solute aggregation inside the vesicles, the absorbance was determined after first disrupting the vesicle membranes by adding Triton X-100 surfactant to each fraction. Encapsulation efficiency (ε) reflects contributions from both the solute in the water pool inside the vesicle and the solute that is electrostatically adsorbed on the vesicle bilayers.

The results of encapsulation experiments using 1 mM CF in V⁺ and V⁻ vesicles are shown in FIG. 6. The left-hand panels show photographs of successive eluted fractions (1.5 ml, each) from the SEC column for V⁺ vesicles (Panel A) and V⁻ vesicles (Panel B). The vesicle-containing fractions are in vials 3-5 (fractions 4-6) in both cases, and this is evident from the high DLS intensity for these samples (plotted as a solid line in the right-hand panels). In addition, the fraction of CF in each vial (from UV-vis) is also plotted as a dotted line in the right-hand panels. Note that vials 3-5 in the case of V⁺ have a strong yellowish tinge, confirming that these vesicles contain an appreciable fraction of CF (23%). On the other hand, vials 3-5 in the case of V⁻ vesicles have a much lower dye content (1.5%). Thus, the anionic CF is efficiently incorporated into the V⁺ vesicles, but not the V⁻ ones. This indicated that the unusually high encapsulation efficiency in V⁺ vesicles was likely due to electrostatic interactions of the CF dye with the vesicles.

Table 2 presents values for apparent encapsulation efficiency (ε) and dye adsorption for CF on egg yolk phosphatidylcholine (EYPC) (phospholipid) vesicles and on V⁺ catanionic vesicles. The ε values were recorded from samples in which the vesicles formed in the presence of the dye and the adsorption values were recorded from samples in which the dye was added to preformed vesicles. In the absence of any specific interactions between the solute and the vesicle wall, ε is a measure of the aqueous volume enclosed by the vesicles relative to the total solution volume. For EYPC vesicles, ε is ca. 1.6%, in agreement with literature values. In comparison, the total enclosed volume of EYPC vesicles calculated from their average DLS radius is about 6%. However, it should be noted that some leakage and rupture of the vesicles is likely to occur during the SEC process, which can explain the difference between these values. Considering next the encapsulation efficiency for the catanionic V⁺ vesicles, we note from Table 1 that their ε is ca. 21%, which is extremely large compared to the EYPC lipid vesicles. Dye encapsulation was evaluated using 1 mM CF since it was found that CF concentrations above 5 mM inhibited vesicle formation. Experiments to measure ε for V⁻ samples were highly irreproducible, yielding ranges from 0 to 3% with no apparent dependence on any governable variables. Given that the total concentration of surfactant is the same for both V⁺ and V⁻ samples, the differences in the value and reliability of ε is unexpected from simple predictions based on enclosed volume. The large and highly reproducible value of ε for the V⁺ samples is likely due to strong, specific interactions between the V⁺ bilayer and the anionic CF dye. If this assertion is correct, one might expect a measurable value for ε even when the dye is added after vesicle formation due to strong interactions of CF with the outer leaflet of the V⁺ bilayer, and this is, in fact, observed as seen by the high adsorption value of CF when it is added to preformed vesicles (Table 1).

TABLE 2

| | Encapsulation | |
|---|---|---|
| | Efficiency, ε | Adsorption |
| EYPC | 1.6 ± 0.2% | 0.40 ± 0.08% |
| V⁺ | 21 ± 2% | 16 ± 4% |

Similar results as for CF, i.e., high encapsulation in V⁺, weak encapsulation in V⁻, were obtained for the other two anionic dyes (LY and SR101) as well. For the cationic solutes (R6G and Dox), the results were switched, i.e., these solutes were efficiently encapsulated in V⁻ samples and weakly in V⁺ samples. Counterparts to FIG. 6 with photographs, DLS intensity, and UV-vis absorbance data, for each of the solutes were obtained. Table 1 shows the ε values (calculated using eq. 1) for each solute in both V⁺ and V⁻ vesicles. It is clear from this data that ionic solutes are efficiently encapsulated in catanionic vesicles having an opposite net charge.

The results presented in Table 1 reflect that the ε values for cationic solutes in V⁻ vesicles are remarkably high: ε is 72% for R6G and 55% for Dox. These values are much higher than those for the anionic solutes in V⁺ vesicles. The reason for this difference may lie in the relative lipophilicities of the counterions for the two surfactants, these being tosylate in the case of CTAT and sodium in the case of SDBS. Tosylate (p-toluene sulfonate) is a hydrophobic counterion, and will mostly (>90%) remain bound to the trimethylammonium headgroup in CTAT, with the aromatic ring of tosylate intercalating into the vesicle bilayer. The bound tosylate counterions will reduce the cationic charge of the bilayer and, in turn, the strength of interactions between anionic solutes and the bilayer will be reduced. In comparison, the sodium counterions in SDBS will be largely dissociated, and therefore the sulfonate headgroups will present a strongly negative bilayer surface for electrostatic binding of cationic moieties.

Regarding solute adsorption, it was found that electrostatic adsorption of CF to the V+ vesicle bilayer made a significant contribution to the apparent encapsulation value, ε. That is, the CF dye was sequestered in CTAT-rich vesicles (V⁺) by two mechanisms: encapsulation in the inner water pool and electrostatic adsorption to the charged bilayer. The overall apparent encapsulation efficiency, ε, was determined to be about 22%. The contribution from electrostatics was obtained by adding the CF to pre-formed V⁺ vesicles, and this resulted in an apparent encapsulation value (ε) value of about 16%, that is, 75% of the encapsulation value obtained when the vesicles were formed in the presence of CF dye as shown in Table 2. That is, electrostatic adsorption contributed about 75% of the overall apparent encapsulation efficiency, ε, value for CF solute in V⁺ vesicles.

Similar experiments conducted with the cationic R6G dye indicated that when the dye was added to pre-made V⁻ vesicles, an εwas obtained that is ca. 85% of the value reported in Table 1. Thus, the electrostatic contribution to solute binding is important for both V⁺ (which strongly bind anionic solutes) and V⁻ vesicles (which strongly bind cationic solutes). That is, excess charge in the bilayer effectively increases the loading capacity of the vesicles. By contrast, the results in Table 1 show that only 0.4% of the dye was adsorbed on the EYPC vesicles, indicating that nonspecific interactions of the dye with the lipid bilayer were weak.

### Example 3

Long-Term Solute Ion Encapsulation and Release in Catanionic Vesicles Formed from Cetyltrimethylammonium Tosylate (CTAT) and Sodium Dodecylbenzenesulfonate (SDBS)

The self-quenching behavior of carboxyfluorescein (CF) was used to monitor dye efflux from vesicles. CF is a widely used probe for vesicle encapsulation due to its ability to undergo efficient self-quenching of fluorescence at millimolar concentrations. For example, when 60 mM CF is entrapped in vesicles, its fluorescence intensity is reduced by 60-80%, but as the dye is released from the vesicle, and thus diluted by the surrounding buffer, its fluorescence intensity increases.

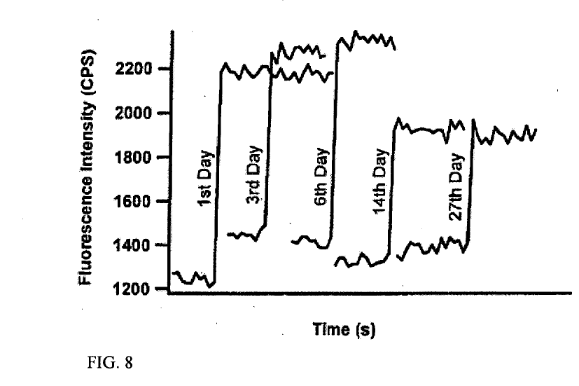

Samples were checked on a specific day by placing a fixed aliquot (1.5 mL) into a 1 cm cuvette and monitoring its emission at 520 nm while exciting at 490 nm using a Spex Fluorolog-3 spectrometer. The intensity was monitored for several minutes to establish the baseline fluorescence intensity, which contains a contribution from both free and encapsulated dyes. After the baseline was established, 100 uL of 10% (w/w) aqueous Triton X-100 was added to disrupt the vesicles. Vesicle disruption results in the release of all dye molecules into solution and a concomitant increase in fluorescence. For these experiments, a substantial volume of sample was prepared on the first day and run on the SEC column to remove free dye. See FIG. 8, which shows several time traces obtained over the course of four weeks from V⁺ vesicles containing encapsulated CF; each trace in FIG. 8 is for data acquired from the same preparation at a given number of days after SEC was run. The traces show the emission intensity before and after the addition of Triton X-100, a nonionic detergent that disrupts both lipid and surfactant vesicles. As can be seen, the resulting release of dye into the solution causes a large jump in emission intensity, and the size of this jump is proportional to the amount of dye encapsulated within the vesicles. We note that the intensity jump reports on the encapsulated dye and not on the adsorbed dye, since addition of Triton X-100 to vesicle samples in which the dye was added after vesicle preparation did not produce an intensity jump. As expected, the largest jump occurs for the freshly prepared vesicle solution where all the dye is encapsulated in the vesicles. We compare the magnitude of the jump on Day x with the highest jump (Day 0) and thereby obtain the fraction of the dye released on day x, R(t=x), the calculation of which is described below. It should be noted that R(t) may actually underestimate the degree of dye retention since it does not account for dequenching occurring within the vesicles as the dye leaks out. This effect will be negligible in the catanionic samples since the dye concentration remains nearly unchanged over the time course of the experiment.

To monitor long-term leakage rates, the fraction of dye released as a function of time, R(t), was calculated for a given day. This quantity measures the fraction of encapsulation on Day x relative to the initial value on Day 0:

$$R ( x ) = 1 - \{ \frac{{F}_{x} ( final ) - {F}_{x} ( initial )}{{F}_{0} ( final ) - {F}_{0} ( initial )} \cdot \frac{{F}_{0} ( final )}{{F}_{x} ( final )} \} \qquad ( 2 )$$

where F(initial) and F(final) are the fluorescence intensities before and after adding the Triton X-100. This approach allows the direct determination of the proportion of the dye released on a daily basis and accounts for deviations due to long-term drift in the spectrophotometer.

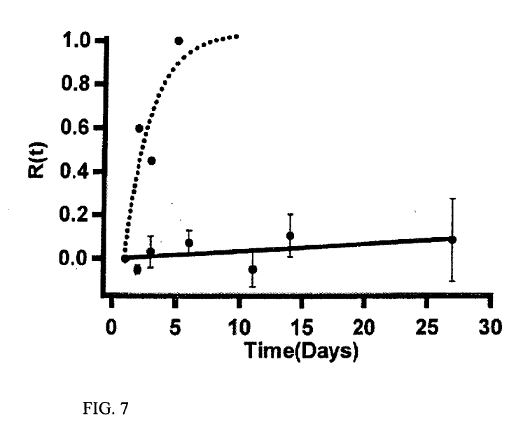

Plots of R(t) are shown in FIG. 7 for CF in V⁺ (solid line) and in EYPC (dotted line) vesicles. The results for vesicles formed from EYPC show that the CF dye is released rapidly over a period of about 5 days, yielding an estimated half-life of ca. 2 days for the entrapped dye. When R(t) reaches 1 there is no longer an increase in fluorescence emission upon addition of detergent, i.e., the dye concentration inside the vesicles has equilibrated with that of the bulk solution. Note that the equilibration takes place by transport across the membrane and not by vesicle degradation, because the vesicles themselves are stable for up to several weeks. In contrast to EYPC vesicle samples, V⁺ samples are able to encapsulate CF over an extremely long period of time. The release of CF is approximately 20% after 27 days giving an estimated half-life of 84 days for the entrapped dye. DLS data taken over the 27-day course of the experiments show that the catanionic vesicle average radii remain unchanged and indicate that vesicle fusion or rupture is not occurring to any significant degree. This indicates a fundamental difference in the permeability of V⁺ membranes to anionic solutes and in the overall vesicle stability compared with lipid vesicles. That is, we found that the release rate of CF from V⁺ catanionic surfactant vesicles was at least 40 times slower than from EYPC vesicles. Thus, the catanionic vesicles (V⁺) achieved much better encapsulation stability than did the EYPC vesicles.

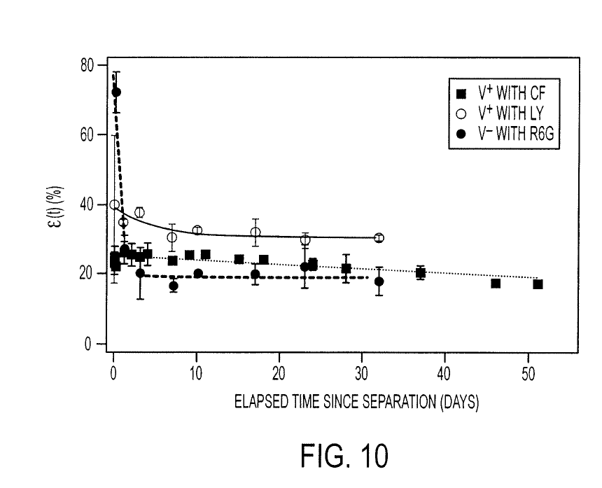

A more general procedure based on SEC that can be applied to a wide range of solutes, including non-self-quenching and non-fluorescent ones was also used. The initial vesicle-solute mixture was purified using SEC (as described above) to remove the free, unencapsulated solute. The sample was then checked for release of solute from the vesicles over the course of several weeks. For this purpose, small-scale separations using quick-spin columns pre-packed with Sephadex G50 (fine) were performed (column from Roche, additional beads for repacking the columns from Sigma). On a specific day, a 100 μL aliquot was run through a quick-spin column by centrifugation (3000 rpm, 15 s), and the eluted fraction was evaluated using UV-vis spectroscopy. Any solute that had been released from the vesicles was retained by the quick-spin column. Therefore, the amount of solute eluted by the column corresponded to the solute still encapsulated by the vesicles. The UV-vis absorption value for the eluted sample was divided by the corresponding value obtained on day zero (immediately after SEC) to yield a fraction of solute that remains encapsulated in the vesicles. This method directly yielded the apparent encapsulation, ε, as a function of time. The procedure was repeated at various times to create a release curve, i.e., encapsulated solute efficiency (ε(t)) vs. time elapsed, as shown in FIG. 10 for three different solute/vesicle combinations. In order to corroborate the release data obtained by this more general procedure, one of the solute/vesicle combinations considered was CF dye in V⁺ vesicles, so that the results could be compared to those obtained with the self-quenching method, described above. FIG. 10 shows that the data for CF in V⁺ vesicles (solid squares) are quite comparable to results for the same CF/V⁺ system obtained using the self-quenching of CF. The data obtained with the more general procedure yields a half life for CF in the vesicles of 114 days, while a more limited data set obtained with the self-quenching method yielded an 84 day half-life. Thus, the results for the CF/V⁺ system obtained with the more general procedure where comparable to the results obtained with the self-quenching method.

Also shown in FIG. 10 are results for Lucifer yellow (LY) in V⁺ vesicles (hollow circles) and Rhodamine 6G (R6G) in V⁻ vesicles (solid circles). The encapsulation efficiency, ε, values for both LY and R6G start out significantly higher than that of CF in V⁺, but decay over the course of a few days to a comparable value of ε (from 0.2 to 0.3). R6G has the largest initial rate of dye leakage; this may be because it is encapsulated to a much greater extent (Table 1) than the other two dyes. On the whole, our new results confirm that oppositely charged solutes can be encapsulated for very long periods of time in catanionic vesicles. For comparison, the half-life for CF in EYPC liposomes is only about 2 days, is which means that the surfactant vesicles retain dye for about 40-60 times as long.

Thus, experiments to determine the encapsulation of the anionic dyes, LY and SR101, and of two cationic solutes, the dye R6G and the anti-cancer drug Dox were performed. The initial value of ε for each of these solutes in both V⁺ and V⁻ vesicles was determined. The encapsulation efficiency, ε, was monitored as a function of time for three different solute/vesicle combinations.

In summary, in the experiments, the apparent encapsulation of several different charged solutes in catanionic CTAT/SDBS vesicles was determined. Solutes were found to be weakly encapsulated by vesicles having like sign of charge as the solute, but contained much more efficiently in vesicles having opposite sign of charge as the solute. Efficient containment in vesicles having opposite charge is understood to be due to strong electrostatic interactions between the solute and the vesicle bilayer. At 1 mM solute concentrations, apparent encapsulation values ranged from 21% to 72%. For example, positively charged catanionic vesicles (V⁺) were found to encapsulate the anionic CF solute with an apparent efficiency of 21%. This high apparent encapsulation efficiency is understood to be the result of electrostatic interaction between the anionic solute and the excess positive charge of the V⁺ bilayer.

Long-term solute release kinetics were monitored for three vesicle/solute preparations. Release profiles show that all dyes are encapsulated for long periods of time. Both R6G, and to a lesser extent LY, have an initial rapid dye release that bring them close to the initial value for CF. The long-term stability of the encapsulation is understood to be due to low membrane permeability. The fusion of catanionic vesicles occurs on a relatively long time scale of months. The encapsulation of anionic solutes does not appear to radically alter this process. Thus catanionic vesicles are promising candidates for high efficiency capture and long-term encapsulation of ionic solutes.

Dynamic light scattering (DLS) and small angle neutron scattering (SANS) techniques were used to measure the effects of solute loading on vesicle integrity and stability. DLS results showed that V⁺ samples appear to undergo an increase in radius when solutes are added at 1 mM, but that the effect on SDBS (V⁻) vesicles is negligible. SANS experiments confirmed that vesicles remain intact when loaded with strongly-interacting probes.

### Example 4

Effect of Solute Ions on the Stability of Catanionic Vesicles Formed from Cetyltrimethylammonium Tosylate (CTAT) and Sodium Dodecylbenzenesulfonate (SDBS)

A low solute concentration, e.g., 1 mM, was used in the experiments to ensure the stability of our vesicle formulations. At concentrations above 5 mM, the solutes seemed to compromise the integrity of the vesicles, as revealed by large changes in vesicle size (from DLS) and/or by the formation of a precipitate over time. Even at a concentration of 1 mM, some solutes may have a large effect on vesicle morphology. To study these aspects in some detail, dynamic light scattering (DLS) and small angle neutron scattering (SANS) were used. DLS was performed on purified vesicles obtained from the SEC column (after removing all the free solute); the sizes of solute-containing vesicles were compared with the sizes of neat vesicles (no solute). DLS gave radii of 74 nm for neat V⁺ vesicles and 70 nm for neat V⁻ vesicles. Passing these neat vesicles through an SEC column changed their sizes slightly and the new radii were 81 nm for V⁺ and 98 nm for V⁻ vesicles. The incorporation of 1 mM solute had a negligible effect on vesicle size in some cases, but a large effect in others (Table 1). For example, both V⁺ and V⁻ vesicle radii were essentially unchanged by 1 mM CF. However, while 1 mM of the anionic solute Lucifer yellow (LY) had no effect on V⁻ vesicles, it induced a 2.5 fold increase in the radii of V⁺ vesicles. Interestingly, the effects on vesicle size seemed to be more significant for V⁺ vesicles than for V⁻, with both cationic and anionic solutes.

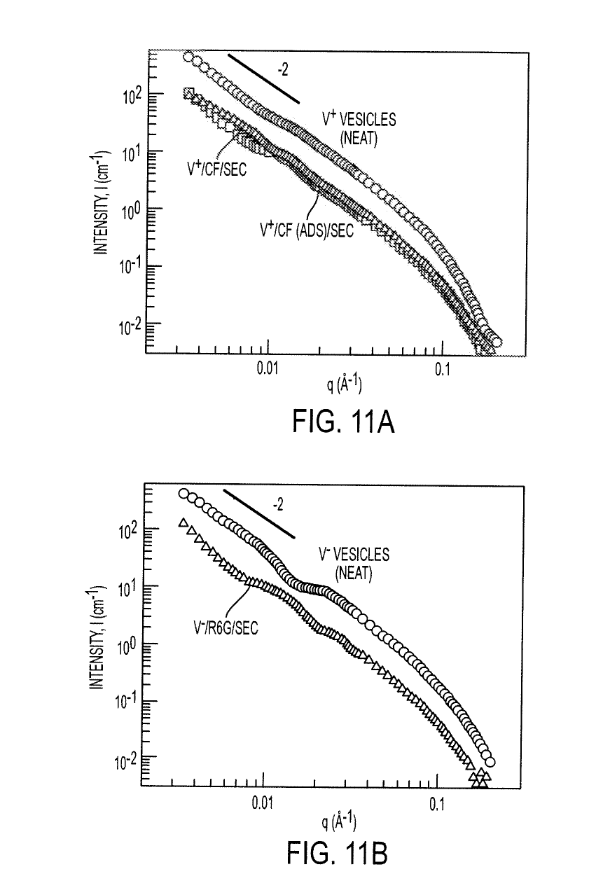

The effects of solutes on catanionic vesicles were also studied using small angle neutron scattering (SANS), which is a sensitive probe of nanoscale structure. SANS data are presented in FIG. 11 for two mixtures of vesicles and oppositely charged solutes: V⁺/CF, and V⁻/R6G. FIG. 11a shows data for the neat V⁺ vesicles with no solute, and for the same vesicles prepared with 1 mM CF and purified by SEC. Additionally, data are shown for a sample of the same vesicles with 1 mM CF added after preparation (i.e., with the dye adsorbed on the bilayers), followed by purification by SEC. Passing the vesicles through SEC lowers the vesicle concentration, which is why the latter two data sets show a lower intensity. Nevertheless, all three curves have approximately the same shape and all show a limiting slope of −2 at low q, which is indicative of scattering from vesicle bilayers. Similar observations also hold for FIG. 11b, which reports data for neat V⁻ vesicles and for the same vesicles with 1 mM R6G followed by SEC. Again, the intensity levels are lower due to the SEC purification, but the −2 slope is maintained. Thus, SANS confirms that all these samples contain intact unilamellar vesicles. In all cases, there appear to be subtle changes in vesicle size and polydispersity upon incorporation of solute.

### Example 5

Separation of Solute Ions with Catanionic Vesicles Formed from Cetyltrimethylammonium Tosylate (CTAT) and Sodium Dodecylbenzenesulfonate (SDBS)

Catanionic vesicles were prepared with equimolar mixtures of two solutes, the cationic organic dye rhodamine 6G (R6G) and the anionic dye carboxyfluorescein (CF). The total solute concentration was maintained at either 0.5 or 1.0 mM, and experiments were done with both positively charged vesicles (V⁺) (excess of cationic CTAT) and negatively charged vesicles (V⁻) (excess of anionic SDBS) formed from cetyltrimethylammonium tosylate (CTAT) and sodium dodecylbenzenesulfonate (SDBS).

Experiments with these solute mixtures were performed and analyzed by performing size exclusion chromatography on the sample and measuring the dye concentration of each fraction to obtain an apparent encapsulation value, ε, for each dye (see below). The dye concentration in each fraction was determined by UV-vis spectroscopy. To account for the overlapping of the dye spectra, we subtracted a scaled spectrum of pure R6G from the total spectrum in order to find the peak absorbance of CF.

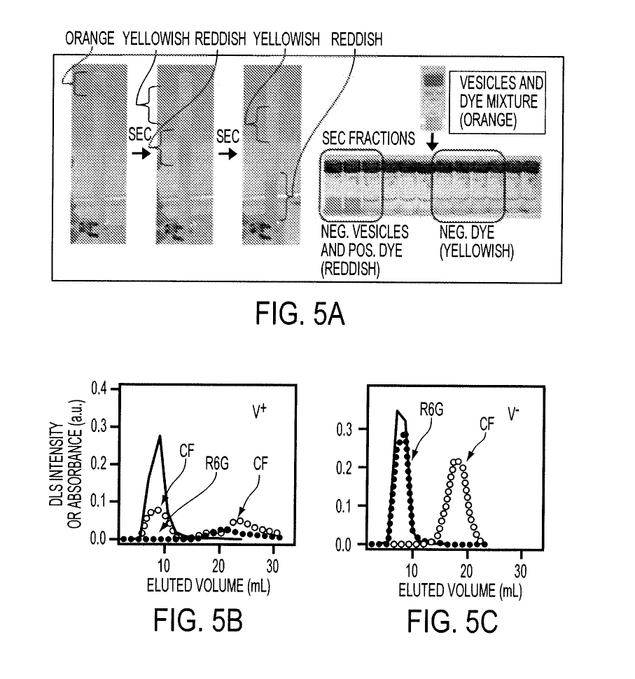

In an experiment, vesicles made from a mixture of cetyltrimethylammonium tosylate (CTAT) and sodium dodecylbenzenesulfonate (SDBS) were added to a solution containing 0.5 M carboxyfluorescein (CF) (anionic dye) and 0.5 M Rhodamine 6G (cationic dye). The vesicles were made from a mixture with an excess of sodium dodecylbenzenesulfonate (SDBS) and possessed a negative net charge. In the mixture the vesicles efficiently sequestered the cationic Rhodamine 6G molecules. The entire mixture was added to a gel filtration column that is packed with sephadex G50 resin, which separates the Rhodamine 6G bearing vesicles from the free carboxyfluorescein (CF). The separation can be clearly seen in FIG. 5.

Results from an experiment with an equimolar mixture of CF and R6G, at a total dye concentration of 0.5 mM, in V⁺ vesicles are shown in FIG. 5, Panels B and C. While 31% of the anionic CF is carried through the SEC column within the V⁺ vesicle band, no detectable R6G emerges with the vesicles. In short, the V⁺ vesicles are able to selectively encapsulate the anionic dye, and thereby separate it from the dye mixture. The opposite behavior is observed for the same dye mixture in V⁻ vesicles (Panels C, D). In this case, the V⁻ vesicle band emerging out of the SEC column contains 88% of the R6G, while the amount of CF in this band is negligible. Thus, the V⁻ vesicles are able to bind and separate the cationic dye from the dye mixture. To our knowledge, this is the first demonstration of using surfactant vesicles as a means to separate ionic compounds. We conducted the same experiments with a total dye concentration of 1.0 mM CF and R6G, and obtained similar encapsulation values.

In another experiment, catanionic vesicles were used to separate the anionic dye Lucifer yellow (LY) and the cationic drug doxirubicin (Dox). The total compound concentration was 1 mM. Very efficient separation was observed, much like in FIG. 5. Thus, highly efficient separations of mixtures of similar sized but oppositely charged probe molecules were performed by using vesicles to control elution time of ionic probe molecules in SEC. Catanionic surfactant vesicles are promising candidates for applications such as separations. A separation can be carried out by determining the sign of charge of a target ion, and producing catanion vesicles having a net surface charge of opposite sign. The catanionic vesicles can be formed in the solution with the ionic probe molecules or can be formed separately and then added to the ionic solution. After the vesicles have sequestered the ions, they can be separated by any techniques for selectively separating vesicles, for example, size exclusion chromatography (SEC), affinity chromatography, or electrokinetic chromatography.

### Example 6

### Catanionic Vesicles for Storage and Controlled Release of Compounds

Important potential applications for catanionic vesicles are in storage or controlled release applications (e.g., in drug delivery, agrochemicals, or cosmetics). This is an area of great promise, as evidenced by the success of the liposome-based delivery of the chemotherapeutic drug, doxorubicin. Most research in this area has focused on phospholipid vesicles (liposomes).

As shown by the results presented herein, catanionic vesicles according to the invention can have loading efficiencies that far surpass the values that can be obtained with liposomes. Moreover, catanionic vesicles according to the invention can retain solutes for 50 times as long as is possible with liposomes. Recent studies by Kuo et al. show catanionic vesicles to be nontoxic towards mouse fibroblast and liver cells. Therefore, catanionic vesicles are an attractive alternative to phospholipid vesicles (liposomes) for many controlled release applications.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A method for sequestering a solute ion within a catanionic vesicle comprising:
determining the charge of the solute ion;
creating a catanionic vesicle having a net surface charge opposite to the charge of the solute ion;
combining the catanionic vesicle with the solute ion; and
allowing the catanionic vesicle to sequester the solute ion.

2. The method of claim 1, wherein the solute ion is in solution, comprising:
adding a cationic surfactant and an anionic surfactant to the solution, in a ratio effective to produce the catanionic vesicles.

3. The method of claim 2, wherein the solute ion is selected from the group consisting of a biologically active compound, a pharmaceutical agent, a fluorescently active chemical, a cosmetic chemical, an agriculturally active chemical, a fertilizer, a nutrient, a pesticide, an herbicide, and combinations.

4. The method of claim 1, wherein the solute ion is in a bulk solution, and the catanionic surfactant vesicle comprises a bilayer comprising a cationic surfactant and an anionic surfactant, and an inner pool separated from the bulk solution by the bilayer, the method comprising:
combining the catanionic surfactant vesicle with the bulk solution and sequestering the solute ion in the inner pool and/or the bilayer of the catanionic vesicle, and
separating the solute ion from the bulk solution by separating the catanionic surfactant vesicle from the bulk solution.

5. The method of claim 4, wherein the solute ion is selected from the group consisting of an atomic ion, a charged inorganic molecule, and a charged organic molecule.

6. The method of claim 4 wherein the separating comprises size exclusion chromatography, affinity chromatography, and/or electrokinetic chromatography.

7. An aqueous composition, comprising,
an aqueous environment and a catanionic surfactant vesicle,
the catanionic surfactant vesicle comprising
a bilayer comprising a cationic surfactant and an anionic surfactant and having a net surface charge;
an inner pool separated from the aqueous environment by the bilayer;
a solute ion having a charge, within the inner pool and/or the bilayer;
the net surface charge of the bilayer being opposite to that of the solute ion.

8. The aqueous composition of claim 7, wherein the solute ion is selected from the group consisting of a metal, carboxyfluorescein, Lucifer yellow, Rhodamine 6G, Sulforhodamine 101, a drug, doxorubicin, a chemotherapeutic agent, a natural product, a peptide, an oligopeptide, a polypeptide, a nucleotide, an oligonucleotide, a polynucleotide, DNA, RNA, derivatives of these, and combinations.

9. The aqueous composition of claim 7, wherein the anionic surfactant is selected from the group consisting of alkyl sulfate, sodium octyl sulfate, sodium decyl sulfate, sodium dodecyl sulfate, sodium tetra-decyl sulfate, alkyl sulfonates, sodium octyl sulfonate, sodium decyl sulfonate, sodium dodecyl sulfonate, alkyl benzene sulfonates, sodium octyl benzene sulfonate, sodium decyl benzene sulfonate, sodium dodecyl benzene sulfonate, fatty acid salt, sodium octanoate, sodium decanoate, sodium dodecanoate, sodium salt of oleic acid, derivatives of these, and combinations.

10. The aqueous composition of claim 7, wherein the cationic surfactant is selected from the group consisting of alkyl trim ethylammonium halide, octyl trimethylammonium bromide, decyl trimethylammonium bromide, dodecyl trimethylammonium bromide, myristyl trimethylammonium bromide, cetyl trimethylammonium bromide, alkyl trimethylammonium tosylate, octyl trimethylammonium tosylate, decyl trimethylammonium tosylate, dodecyl trimethylammonium tosylate, myristyl trimethylammonium tosylate, cetyl trimethylammonium tosylate, N-alkyl pyridinium halide, decyl pyridinium chloride, dodecyl pyridinium chloride, cetyl pyridinium chloride, derivatives of these and combinations.

11. The aqueous composition of claim 7, wherein the cationic and/or the anionic surfactant is selected from the group consisting of SDS, DTAC, DTAB, DPC, DDAO, DDAB, SOS, AOT, derivatives of these, and combinations.

12. The aqueous composition of claim 7, wherein the cationic surfactant is a single alkyl chain surfactant and/or the anionic surfactant is a single alkyl chain surfactant.

13. The aqueous composition of claim 7,
wherein the solute ion is a cation having positive charge and
wherein the cationic surfactant and anionic surfactant comprising the bilayer are in proportions creating a bilayer with a negative net surface charge.

14. The aqueous composition of claim 7,
wherein the solute ion is an anion having negative charge and
wherein the cationic surfactant and anionic surfactant comprising the bilayer are in proportions creating a bilayer with a positive net surface charge

15. The aqueous composition of claim 7,
wherein the bilayer comprises cationic surfactant and anionic surfactant in a molar ratio in a range of from about 1:9 to about 9:1, excluding a molar ratio of about 1:1.

16. The aqueous composition of claim 7, wherein the combined weight percentage of cationic surfactant and anionic surfactant in the external aqueous environment is less than about 5 wt %.

17. The aqueous composition of claim 7, wherein the combined weight percentage of cationic surfactant and anionic surfactant in the external aqueous environment is less than from about 0.0001 wt % to about 3 wt %.

18. The aqueous composition of claim 7, wherein the combined weight percentage of cationic surfactant and anionic surfactant in the external aqueous environment is from about 0.5 wt % to about 2 wt %.

19. The aqueous composition of claim 7, wherein the concentration of the solute ion within the catanionic vesicle is greater than the concentration of the solute ion in the aqueous environment.

20. The aqueous composition of claim 7, wherein the encapsulation efficiency of the solute ion in the vesicle is at least about 2%.

21. The aqueous composition of claim 7, wherein the percentage of solute adsorbed on the bilayer is at least about 0.5%.

22. A method of introducing an agent into a cell, comprising:
contacting the cell with a composition comprising catanionic surfactant vesicles comprising a bilayer of a cationic surfactant and an anionic surfactant defining an inner pool comprising the agent, the net surface charge of the bilayer being opposite to that of the agent.

23. The method of claim 22, wherein the cell is within a living organism.

24. The method of claim 22, wherein the composition comprising the catanionic surfactant vesicles is administered orally or intravenously.

25. A method of introducing a nucleic acid into a cell, comprising:
administering catanionic surfactant vesicles comprising a nucleic acid to the cell,
wherein the catanionic surfactant vesicle comprises
a bilayer comprising a cationic surfactant and an anionic surfactant,
an inner pool separated from an aqueous environment by the bilayer,
the inner pool and/or the bilayer comprising the nucleic acid,
the nucleic acid having a negative charge,
the cationic surfactant and anionic surfactant comprising the bilayer having a net positive surface charge.

26. A kit, comprising:
a premeasured amount of an anionic surfactant in a first labeled container; and
a premeasured amount of a cationic surfactant in a second labeled container,
wherein the premeasured amount of the anionic surfactant and the premeasured amount of the cationic surfactant are selected so that when the premeasured amount of the anionic surfactant and the premeasured amount of the cationic surfactant are added to a predetermined amount of water containing a solute ion having a charge, catanionic surfactant vesicles are formed and
wherein the catanionic surfactant vesicles comprise
a bilayer comprising a cationic surfactant and an anionic surfactant and having a net surface charge,
an inner pool separated from the aqueous environment by the bilayer,
the solute ion within the inner pool and/or the bilayer, and
the net surface charge of the bilayer being opposite to that of the solute ion.

27. A kit, comprising:
a mixture of an anionic surfactant and a cationic surfactant in a labeled container,
wherein the anionic surfactant and the cationic surfactant are in a predetermined molar ratio in the mixture,
wherein the predetermined molar ratio is selected so that when the mixture is added to a predetermined amount of water containing a solute ion having a charge, catanionic surfactant vesicles are formed and
wherein the catanionic surfactant vesicles comprise
a bilayer comprising a cationic surfactant and an anionic surfactant and having a net surface charge,
an inner pool separated from the aqueous environment by the bilayer,
the solute ion within the inner pool and/or the bilayer, and
the net surface charge of the bilayer being opposite to that of the solute ion.

28. A kit, comprising:
a mixture of an anionic surfactant, a cationic surfactant, and a solute ion having a charge in a labeled container,
wherein the anionic surfactant and the cationic surfactant are in a predetermined molar ratio in the mixture,
wherein the predetermined molar ratio is selected so that when the mixture is added to a predetermined amount of water, catanionic surfactant vesicles are formed and
wherein the catanionic surfactant vesicles comprise
a bilayer comprising a cationic surfactant and an anionic surfactant and having a net surface charge,
an inner pool separated from the aqueous environment by the bilayer,
the solute ion within the inner pool and/or the bilayer, and
the net surface charge of the bilayer being opposite to that of the solute ion.
